(19) United States
(12) Patent Application Publication
(10) Pub. No.: US 2026/0096983 A1
(43) Pub. Date: Apr. 9, 2026

(54) Thermosensitive hydrogel composition

(71) Applicant: Desentum Oy, Espoo (FI)

(72) Inventors: Jaana Haka, Espoo (FI); Heidi Heiniluoto, Espoo (FI); Kristiina Takkinen, Espoo (FI); Juha Rouvinen, Espoo (FI); Silja Halme, Espoo (FI); Kati Sallinen, Espoo (FI)

(21) Appl. No.: 19/115,261

(22) PCT Filed: Sep. 26, 2023
(86) PCT No.: PCT/FI2023/050544

§ 371 Date: Mar. 26, 2025

(30) Foreign Application Priority Data
Sep. 26, 2022 (FI) 20225835

(51) Int. Cl. A61K 9/00; A61K 9/06; A61K 39/35; A61K 39/36; A61K 39/395; A61K 47/10

(52) U.S. Cl. CPC A61K 9/0024; A61K 9/06; A61K 39/35; A61K 39/36; A61K 39/395; A61K 47/10

(57) ABSTRACT

According to an example aspect of the present invention, there is provided a biocompatible thermosensitive hydrogel composition for subcutaneous or intramuscular injection. The hydrogel comprises a poloxamer, wherein said poloxamer is Poloxamer 338, Poloxamer 188 or Poloxamer 407 or a mix thereof. Said composition comprises a therapeutic protein or antigen embedded in the composition for sustained release.

### FIELD

The present disclosure belongs to the field of thermosensitive hydrogels. More specifically, the present disclosure relates to local delivery of therapeutic proteins or antigens through thermosensitive hydrogels, their composition materials, formulations, application methods, and usage.

### BACKGROUND

Allergic diseases, for example, asthma, rhinitis, eczema and food allergies are reaching epidemic proportions in the world. The hypersensitive reactions of these diseases are based on the formation of immunoglobulin E (IgE) antibodies against, in principle, harmless protein antigens, allergens. Treatment of allergic diseases by administering patients with a hypoallergenic variant of a desired allergen has been suggested, for instance, in WO2009153414.

The trends in the treatment of all allergic symptoms have recently been towards an active induction of tolerance using allergen-specific desensitization instead of avoiding the allergen, which is often not possible, or merely treating the symptoms. Current desensitization therapy is based on allergens purified from natural sources, wherein batch-to-batch variations both in amounts of allergen components as well as the presence of other non-allergen protein substances may lead to problems related to finding and maintaining the right dosage and efficiency of the treatment. These problems lead to a potential risk of anaphylactic side effects and sensitization to new allergens (neosensitization). The use of recombinant hypoallergen variants for desensitizing would remove the disadvantages related to batch-to-batch variations and minimize the amount of other protein components which may cause side-effects or unwanted activation of immune system towards these impurity proteins.

WO2012143374 discloses mutant polypeptides useful as hypoallergens. The recombinant birch pollen Bet v 1 polypeptides were produced containing mutations at selected amino acid positions to reduce or fully diminish their ability to trigger sensitized mast cells or basophils leading to various allergic reactions but to retain their capacity to induce the production of protective IgG antibodies.

WO2019135027 discloses modified Equ c 1 polypeptides and the use of such polypeptides as hypoallergenic variants for desensitizing against horse allergy.

For the treatment of allergies, allergen-specific immunotherapy with hypoallergenic variants has the potential of restoring lasting immunological tolerance, however, improved tolerance-promoting dosage formulations are needed to increase the therapeutic efficacy and to decrease side-effects of this approach.

### SUMMARY

According to a first aspect of the present invention, there is provided a biocompatible thermosensitive hydrogel composition for subcutaneous or intramuscular injection. The composition comprises a poloxamer, wherein said poloxamer is Poloxamer 338 or a mix thereof with Poloxamer 188, or said poloxamer is a mix of Poloxamer 407 and Poloxamer 188. The said composition contains up to 25% (w/w) of poloxamer so that the composition comprises 15-20% (w/w) of Poloxamer 338 or Poloxamer 407. The composition comprises a therapeutic protein or antigen embedded in the composition. Preferably, the composition comprises two or more therapeutic proteins or antigens.

According to a second aspect of the present invention, there is provided a method of treating an allergy or an autoimmune disease comprising administering the biocompatible thermosensitive hydrogel composition to a patient in need thereof. The administration is performed by a subcutaneous or intramuscular injection.

According to a third aspect of the invention, there is provided the use of the biocompatible thermosensitive hydrogel composition for the manufacture of a medicament for the treatment of an allergy or an autoimmune disease.

The invention is defined by the features of the independent claims. Some specific embodiments are defined in the dependent claims.

### BRIEF DESCRIPTION OF THE DRAWINGS

FIG. 1. (A) Dissolution of hydrogel compositions comprising hypoallergen DM-101, and (B) DM-101 release from the hydrogel composition comprising 5% P188 and 18% P407. Hydrogel composition of 10% P188 and 20% P407 was used as a control. Released protein was determined with ELISA. Hypoallergen DM-101 contains two modifications (N28K and E101K) in its amino acid sequence as compared to the wild-type birch allergen Bet v 1 (Bet v 1.0101), see WO2012143374.

FIG. 2. Reversibility capability of hydrogel compositions. (A) Gel dissolution time and (B) DM-101 release from the 5% P188 and 18% P407 hydrogel composition was measured after three sol-gel cycles.

FIG. 3. Stability of correctly folded hypoallergenic birch allergen DM 101 in plain buffer and in different mixtures of poloxamers. The amount of DM101 was measured from the solution after storage at +4 and room temperature for the indicated times (0-6 months), and the measurement was performed with an immunoassay which is fold sensitive, i.e. measures only correctly folded DM 101. (A-E) Gel dissolution times and (F-J) DM-101 release.

FIG. 4. Characteristics of P188/P338 hydrogel composition. (A) Dissolution time and (B) DM-101 release from hydrogels 5% P188/18% P407, 2% P188/18% P388 and 0% P188/16% P338.

FIG. 5. Viscosity of P188/P338 hydrogel composition. Viscosity of different hydrogel compositions was determined. The values on X-axis relates to the percentage (w/w) of poloxamers that are named below the values.

FIG. 6. Skin prick test in mice. Darker colour (i.e. Evans blue dye) visualizes inflammation of the skin. To determine the impact of different poloxamer formulations on the allergenicity of DM-101 mice were sensitized to the Birch allergen. A set of mice were intraperitonially sensitized with wild type Bet v 1 and an alum adjuvant. After successful sensitization, i.e. showing a positive IgE response, mice were challenged with different formulations of DM 101 using wild type Bet v 1 as positive control or plain buffer as negative control. Dose 30 μl of 20 mg of DM-101/ml solutions. Results shown are after 30 minutes of intradermal injection. Formulations: F1: DM-101 in the Diluent buffer (10 mM Na₂HPO₄, 1.8 mM KH₂PO₄, 137 mM NaCl, 2.7 mM KCl, pH 7.4); F2: DM-101 in 0.05% P188 in the Diluent buffer; F3: DM-101 in 5% P188/18% P407 in the Diluent buffer; F4: DM-101 (1 mg/ml) with aluminium adjuvant (5 mg/ml) in the Diluent buffer; PBS: negative control, Bet v 1: positive control containing wild-type birch allergen. C48/80: mast cell degranulation compound 48/80 (20 mg/mL; Sigma) as a positive control. Results: F3 showed mildest reaction in all mice. Positive skin responses were seen with F1 and F2. Positive skin responses were also seen with F4 but these were milder than with F1 and F2 but more noticeable than with F3.

FIG. 7. Body temperature after subcutaneous injection. The impact of the poloxamer formulations on the anaphylactic potency of DM-101 is clearly demonstrated. Both poloxamer formulation candidates decrease the anaphylactic potency of DM-101 approximately 4-fold. Differences between the 5% P188/18% P407 and 16% P338 formulations are minor. Body temperature after subcutaneous injection. (A) Temperature curves of control animals and allergic animals challenged with Formulation 1. (B) Temperature curves of allergic animals challenged with Formulations 3 and 5.

FIG. 8. Release of allergens beta-lactoglobulin (BLG) and recombinant Ara h 2 from 5% P188/18% P407 and 16% P338 hydrogels. Amount of the released protein was determined with o-pthalaldehyde (OPA) assay according to the instructions of the manufacturer (Thermo Scientific). BLG behaves much the same as DM-101, but the total amount of released allergen determined for Ara h 2 remains somewhat lower.

### EMBODIMENTS

The present invention discloses a thermosensitive hydrogel composition for subcutaneous or intramuscular injection. The composition comprises a therapeutic protein or an antigen. The release of the therapeutic protein or antigen is controlled by the hydrogel composition.

### Thermosensitive Biocompatible Hydrogel

The term “biocompatible hydrogel” means a hydrogel that does not produce toxic (or cytotoxic) or harmful effects or products and is not immunogenic itself. This is essential so that during the treatment, the hydrogel itself does not induce a rejection response.

The composition comprises poloxamers, which are nonionic triblock copolymers composed of a central hydrophobic chain of polyoxypropylene (poly(propylene oxide)) flanked by two hydrophilic chains of polyoxyethylene (poly(ethylene oxide)). Because the lengths of the polymer blocks can be customized, many different poloxamers exists. Poloxamers are sold under tradenames Pluronic, Synperonic and Kolliphor. In some embodiments, the composition comprises Poloxamer 338 (P338, Pluronic F108). In other embodiments, the composition comprises a mix of Polaxamer 388 with Poloxamer 188 (P188, Pluronic F68). In yet other embodiment, the poloxamer is a mix of Poloxamer 407 (P407, Pluronic F127) and Poloxamer 188. Said composition contains up to 15%, 18%, 20%, 22%, 25% (w/w) of poloxamer so that the composition comprises 15%, 16%, 17%, 18%, 19%, 20% (w/w) of Poloxamer 338 or Poloxamer 407.

In some embodiments, the biocompatible hydrogel composition comprises 4-6% (w/w) of Poloxamer 188 and 17-19% (w/w) of Poloxamer 407, preferably 5% (w/w) of Poloxamer 188 and 18% (w/w) of Poloxamer 407. In other embodiments, the biocompatible composition comprises 15-19% (w/w) of Poloxamer 338, preferably 15-17% (w/w) of Poloxamer 338, more preferably 16% (w/w) of Poloxamer 338. Yet in other embodiments, the biocompatible hydrogel composition comprises 1-3% (w/w) of Poloxamer 188 and 15-19% (w/w) of Poloxamer 338, preferably 2% (w/w) of Poloxamer 188 and 18% (w/w) of Poloxamer 338.

The term “thermosensitive” refers to compositions in which the physical state of the composition depends on temperature. In some embodiments of the present disclosure, the poloxamer hydrogel is at liquid state at temperatures ranging from 4° C. to 25° C. and forms a gel at 30-37° C. Gelation time is less than 150 seconds at 37° C. Preferably a gel is formed in 60 seconds, more preferably in 30 seconds, at 37° C. In some preferred embodiments, the hydrogels are at liquid state at storage temperature ranging from 4° C. to 8° C. In other preferred embodiments, the hydrogels are at liquid state at room temperature 20-25° C.

Thermosensitive behaviour of the hydrogel allows easy injection of the composition at room temperature and formation of an in situ hydrogel implant at 37° C. In situ gel forming drug delivery systems provide a means by which a controlled release depot can be physically inserted into a target site without the use of surgery. These systems avoid the use of large needles or microsurgery and they are injected as low viscous solutions and transform in the body to a gel or solid depot.

### Therapeutic Proteins and Antigens

The biocompatible hydrogel composition of the present disclosure comprises at least one therapeutic protein or antigen embedded in the composition. As used herein, the term “antigen” includes a moiety or a molecule that contains an epitope to which a binding agent (e.g. antibody) can bind to. The term “epitope” is a well-known term in the art and refers to a specific region of an antigen to which an agent (e.g. antibody) can bind. An epitope can be linear, conformational, non-linear or discontinuous epitope. In the case of polypeptide antigens, it will be appreciated by a person skilled in the art that the presence of an epitope may or may not be dependent on the secondary, tertiary or quaternary structure of the polypeptide. For example, in some embodiments an agent can bind certain amino acid sequence independent of the folding of the said sequence. In other embodiments, an agent binds the epitope only if it has a certain three-dimensional structure.

In some embodiments, up to 1%, 2%, 5%, 8%, 10%, 12%, 15%, 16%, 17%, 18%, 19%, 20% of the therapeutic protein or antigen is released from the composition in 30 minutes at 37° C. The release is determined as percentage of protein in the sample in relation to the starting composition at the specified time point. This can be determined for example by enzyme-linked immunosorbent assay, SDS-PAGE, protein concentration measurements or mass spectrometry or with any other suitable method for determining protein content in the sample. In other embodiments, at least 55%, 60%, 65%, 70%, 75%, 80%, 85%, 90%, 95%, 98%, 99% of the therapeutic protein or antigen is released from said composition in 300 minutes, preferably in 240 minutes, more preferably in 180 minutes at 37° C.

The advantage of such timed release is that a protein or antigen that potentially causes an adverse immune response can be presented to the immune system in a controlled manner. On one hand, the protein or antigen is released from the composition fast enough for immune system activation in a time frame where the patient can be monitored by healthcare professionals, and on the other hand, the protein is not released from the composition too fast, which could lead to adverse immune response.

In some embodiments, the embedded therapeutic protein is an allergen, preferably a hypoallergen, more preferably a genetically engineered hypoallergen. The allergen can be a purified protein, a recombinant protein, a pollen extract or an extract from animal hair, dander, saliva or urine.

In some embodiments, the allergen is a plant pollen, or a genetically engineered hypoallergen thereof. Such plant pollens include but are not limited to pollens from trees, such as birch, alder, ash, aspen, cedar, juniper, maple, olive or oak; from grass, such as rye or timothy; or from weeds such as mugwort or ragweed. In preferred embodiments, the allergen is pollen from birch or timothy, or a genetically engineered hypoallergen thereof. In other embodiments, the allergen is an animal protein, or a genetically engineered hypoallergen thereof. Such animal proteins include but are not limited to proteins from horse, dog, cat, rodents and rabbit. In preferred embodiments, the allergen is from horse or a genetically engineered hypoallergen thereof.

In preferred embodiments, the allergen is birch allergen Bet v 1, or a genetically engineered hypoallergen thereof. In other preferred embodiments, the allergen is horse allergen Equ c 1, peanut allergen Ara h 2, or a genetically engineered hypoallergen thereof.

In other embodiments, the therapeutic protein or antigen is an autoantigen. The autoantigens include one or more purified natural autoantigens, one or more recombinant autoantigens or derivatives thereof, and one or more fragments of natural or recombinant autoantigens. As used herein, the term “autoantigen” (also known as self-antigen) is an antigen that despite being a normal tissue constituent is the target of a humoral or cell-mediated immune response, as in autoimmune diseases. The autoantigen may, for example, be a protein, a complex of proteins, DNA or RNA, either single- or double-stranded or a glycoprotein. Examples of autoantigens include but are not limited to insulin, proinsulin, glutamic acid decarboxylase, myelin basic protein (MBP), collagen type II, thyroid peroxidase or retinol binding protein-3 (RBP-3).

As used herein, the term “fragment” includes native polypeptides (either degradation products, synthetically synthesized peptides or recombinant peptides) and modified peptides, which may have, for example, modifications rendering the peptides more stable or less immunogenic. Such modifications include, but are not limited to, cyclization, N-terminus modification, C-terminus modification, peptide bond modification, backbone modification and residue modification. The fragment may also comprise further elongations, deletions, substitutions or insertions. The term “peptide” refers herein to any chain of amino acid residues, regardless of its length or post-translational modification (e.g., glycosylation or phosphorylation).

### Use of the Biocompatible Hydrogel Composition

The biocompatible hydrogel composition of the present disclosure allows sustained local delivery of therapeutic proteins or antigens. Sustained delivery results in prolonged and slowed down presentation of said proteins or antigens. Hydrogels can serve as a depot for sufficient quantities of allergens or autoantigens, or fragments thereof, tolerance-promoting adjuvants and optionally tolerance-promoting active immune modulators. The aim is to present the protein or antigen to the immune system in a controlled way to avoid adverse immune response. To further avoid adverse immune responses, the hydrogel composition can also comprise a further compound or agent making allergy-related immune cells less sensitive to stimulation by allergens. Examples of such compounds or agents are antihistamin and anti-IgE antibodies such as Omalizumab.

Further, the hydrogel composition can protect the therapeutic protein or antigen from enzymatic decomposition in the body. Therefore, the hydrogel composition of the present disclosure can allow administration of sensitive molecules, such as RNA.

In some embodiments, the biocompatible hydrogel composition is for use in allergen-tolerance promoting immunotherapy or treatment of an autoimmune disease. Autoimmune diseases include but are not limited to diseases such as type I diabetes, rheumatoid arthritis, autoimmune uveitis and multiple sclerosis. In other embodiments, the biocompatible hydrogel composition is for use in the treatment of allergic diseases, such as allergic conjunctivitis, allergic rhinitis, and allergic asthma.

In some embodiments, the antigen comprises live virus, live bacteria, killed virus, killed bacteria, nucleic acids, protein subunits of infectious agents, or mixtures thereof. In preferred embodiments, the hydrogel composition of the present disclosure is a vaccine.

The biocompatible hydrogel composition comprises in some embodiments a sterile diluent such as water for injection, saline solution, fixed oils, polyethylene glycols, glycerine, propylene glycol or other synthetic solvents, antioxidants such as ascorbic acid or sodium bisulfite, chelating agents such as ethylenediaminetetraacetic acid (EDTA), preservatives, buffers such as acetates, citrates or phosphates.

The present disclosure further provides a method of preventing or treating an allergy or an autoimmune disease comprising administering the biocompatible thermosensitive hydrogel composition to a patient in need thereof. The administration is preferably performed by a subcutaneous or intramuscular injection. Subcutaneous injections are preferably performed at one-week or two-week intervals as long as needed.

As used herein, the terms “subject,” and “patient,” are used interchangeably herein to refer to an animal being treated with one or more exemplary compounds as taught herein, including, but not limited to, simians, humans, avians, felines, canines, equines, rodents, bovines, porcines, ovines, caprines, mammalian farm animals, mammalian sport animals, and mammalian pets. A suitable subject for various embodiments can be any animal, including a human, that is suspected of having, has been diagnosed as having, or is at risk of developing a disease that can be ameliorated, treated or prevented by administration of one or more exemplary compounds as described herein.

The present disclosure further provides use of the biocompatible thermosensitive hydrogel composition for the manufacture of a medicament for the prevention or treatment of an allergy or an autoimmune disease.

In one embodiment, the hydrogel comprises 5% of P188 and 18% P407 in diluent with 0.2 mg/mL DM-101 in 10 mM Na₂HPO₄, 1.8 mM KH₂PO₄, 137 mM NaCl, 2.7 mM KCl, pH 7.4.

In another embodiment, the hydrogel comprises 16% P338 in diluent with 0.2 mg/mL DM-101 in 10 mM Na₂HPO₄, 1.8 mM KH₂PO₄, 137 mM NaCl, 2.7 mM KCl, pH 7.4.

Unless otherwise stated, properties that have been experimentally measured or determined herein have been measured or determined at room temperature. Unless otherwise indicated, room temperature is 25° C.

It is to be understood that the embodiments of the invention disclosed are not limited to the particular structures, process steps, or materials disclosed herein, but are extended to equivalents thereof as would be recognized by those ordinarily skilled in the relevant arts. It should also be understood that terminology employed herein is used for the purpose of describing particular embodiments only and is not intended to be limiting.

Reference throughout this specification to “one embodiment” or “an embodiment” means that a particular feature, structure, or characteristic described in connection with the embodiment is included in at least one embodiment of the present invention. Thus, appearances of the phrases “in one embodiment” or “in an embodiment” in various places throughout this specification are not necessarily all referring to the same embodiment.

As used herein, a plurality of items, structural elements, compositional elements, and/or materials may be presented in a common list for convenience. However, these lists should be construed as though each member of the list is individually identified as a separate and unique member. Thus, no individual member of such list should be construed as a defacto equivalent of any other member of the same list solely based on their presentation in a common group without indications to the contrary. In addition, various embodiments and example of the present invention may be referred to herein along with alternatives for the various components thereof. It is understood that such embodiments, examples, and alternatives are not to be construed as defacto equivalents of one another, but are to be considered as separate and autonomous representations of the present invention.

Furthermore, the described features, structures, or characteristics may be combined in any suitable manner in one or more embodiments. In the following description, numerous specific details are provided, such as examples of lengths, widths, shapes, etc., to provide a thorough understanding of embodiments of the invention. One skilled in the relevant art will recognize, however, that the invention can be practiced without one or more of the specific details, or with other methods, components, materials, etc. In other instances, well-known structures, materials, or operations are not shown or described in detail to avoid obscuring aspects of the invention.

While the forgoing examples are illustrative of the principles of the present invention in one or more particular applications, it will be apparent to those of ordinary skill in the art that numerous modifications in form, usage and details of implementation can be made without the exercise of inventive faculty, and without departing from the principles and concepts of the invention. Accordingly, it is not intended that the invention be limited, except as by the claims set forth below.

The verbs “to comprise” and “to include” are used in this document as open limitations that neither exclude nor require the existence of also un-recited features. The features recited in depending claims are mutually freely combinable unless otherwise explicitly stated. Furthermore, it is to be understood that the use of “a” or “an”, i.e. a singular form, throughout this document does not exclude a plurality.

### EXPERIMENTAL SECTION

DM-101 Formulation 1. 125, 250 or 500 μg/ml of DM-101 in the Diluent buffer (10 mM Na₂HPO₄, 1.8 mM KH₂PO₄, 137 mM NaCl, 2.7 mM KCl, pH 7.4).

DM-101 Formulation 3. 250, 500, 1000, 2000, or 4000 μg/ml of DM-101 in 5% P188/18% P407 in the Diluent buffer (10 mM Na₂HPO₄, 1.8 mM KH₂PO₄, 137 mM NaCl, 2.7 mM KCl, pH 7.4).

DM-101 Formulation 5. 250, 500, 1000, 2000 or 4000 μg/ml of DM-101 in 16% P338 in the Diluent buffer (10 mM Na₂HPO₄, 1.8 mM KH₂PO₄, 137 mM NaCl, 2.7 mM KCl, pH 7.4).

Ara h 2/BLG in 5% P188/18% P407, 3 ml total volume. 0.3 ml Beta-lactoglobulin, BLG, Sigma, L3908 (2 mg/ml) or peanut allergen rAra h 2 purified as described by Storni et al. 2020 (2 mg/ml), 0.5 ml 30% P188, and 1.8 ml 30% P407 in 0.4 ml of the Diluent buffer (10 mM Na₂HPO₄, 1.8 mM KH₂PO₄, 137 mM NaCl, 2.7 mM KCl, pH 7.4).

Ara h 2/BLG in 16% P338, 3 ml total volume. 0.3 ml Beta-lactoglobulin, BLG, Sigma, L3908 (2 mg/ml) or rAra h 2 purified as described by Storni et al. 2020 (2 mg/ml), and 1.6 ml 30% P338 in 1.1 ml of the Diluent buffer (10 mM Na₂HPO₄, 1.8 mM KH₂PO₄, 137 mM NaCl, 2.7 mM KCl, pH 7.4).

Example 1. 30% (w/w) P407 stock solution was prepared in diluent (10 mM Na₂HPO₄, 1.8 mM KH₂PO₄, 137 mM NaCl, 2.7 mM KCl, pH 7.4) in total volume of 200 mL as follows: 140 g of diluent was weighted to a sterile Schott bottle, 60 g of P407 was weighted and added into the bottle containing the diluent in small batches under constant stirring, the solution was mixed at 4° C. until the poloxamer was completely dissolved.

30% (w/w) P188 stock solution was prepared in diluent (10 mM Na₂HPO₄, 1.8 mM KH₂PO₄, 137 mM NaCl, 2.7 mM KCl, pH 7.4) in total volume of 100 mL. 70 g of the diluent was weighted in a sterile Schott bottle. 30 g of P188 was weighted into the bottle. The solution was mixed at RT until poloxamer was completely dissolved.

Hydrogel with 5% (w/w) of P188, 18% (w/w) of P407 and 0.2 mg/mL DM-101 was prepared in total volume of 100 mL. 60 mL of cold 30% (w/w) P407 stock was added to a sterile Schott bottle. 16.7 mL of cold 30% (w/w) P188 stock solution was added to the bottle. 3.34 mL of cold diluent was then added. Finally 20 mL of 1 mg/mL DM-101 solution was added. The solution was mixed by inverting the bottle or by gentle vortexing.

Example 2. 30% (w/w) P338 stock solution was prepared in diluent (10 mM Na₂HPO₄, 1.8 mM KH₂PO₄, 137 mM NaCl, 2.7 mM KCl, pH 7.4) in total volume of 200 mL as follows: 140 g of diluent was weighted to a sterile Schott bottle, 60 g of P407 was weighted and added into the bottle containing the diluent in small batches under constant stirring, the solution was mixed at 4° C. until the poloxamer was completely dissolved.

Hydrogel with 16% (w/w) of P338 and 0.2 mg/mL DM-101 was prepared in total volume of 93.75 mL. 50 mL of cold 30% (w/w) P338 stock was added to a sterile Schott bottle. 25 mL of cold diluent was added to the bottle. Finally 18.75 mL of 1 mg/mL DM-101 solution was added. The solution was mixed by inverting the bottle or by gentle vortexing.

Gelling time. The samples were incubated at room temperature for 30 minutes. 0.5 mL of sample was taken with a 25 G needle into 1 mL syringe and injected to a glass vial. The vial was placed in water bath at 33° C. or 37° C. and gelling was monitored in 10 second intervals. The time at which the solutions do not move even if the vial is overturned is considered as the gelling time.

Viscosity. Viscosity was measured with a rotational rheometer (AR-G2, TA instruments). Viscosity was measured at 22° C. as function of shear rate in the range 0.5-500 s⁻¹, 5 points per decade.

DM-101 release from hydrogel. Samples were placed in water bath at 37° C. until a clear gel was formed. 150 μl of diluent was carefully added over the surface of the gel. At 30 minutes intervals the diluent was remove and the amount of the release medium and weight the vial was measured. At each time point, fresh diluent (150 μl) was added over the surface of the gel. Sampling was continued until the gel was completely dissolved. Dissolution was estimated by visual inspection. DM-101 was determined from the samples with sandwich type Bet v 1 ELISA according to manufacturer's protocol (Bet v 1 ELISA 2.0EP, Indoor Biotechnologies). Samples were diluted to the standard range of 0.19-100 ng/ml.

Reversibility capability of the hydrogel. 0.5 mL samples were incubated at 37° C. for 15 min for formation of the gel and moved to 4° C. for 15 min for bringing the sample back to solution phase. This cycle was repeated three times before analyzing the release of DM-101 as disclosed above.

Skin prick test. Skin prick test was performed as described by Schöll et al. (2004).

Viscosity of hydrogel formulated DM-101. The aim was to test different P188 and P407 combinations to form hydrogel in order to decrease the viscosity of the solution while maintaining the adequate gelling temperature (T_{sol-gel}). The results are shown in Table 1.

TABLE 1

| Tube label | Hydrogel | Sample | Gelling time | |
|---|---|---|---|---|
| (T_{sol-gel} ° C.*) | composition | volume (ml) | at +37° C. (s) | Notes |
| A | 10% P188 | 0.05 | 10 | Hydrogel composition A. |
| (31.68 ± 0.28) | 20% P407 | 0.1 | 10 | |
| | | 0.2 | 20 | |
| | | 0.4 | 20 | |
| | | 0.8 | 20 | |
| B | 10% P188 | 0.05 | 20 | No remarkable difference to |
| (35.70 ± 0.25) | 18% P407 | 0.1 | 20 | Hydrogel composition A on |
| | | 0.2 | 30 | syringeability. |
| | | 0.4 | 40 | Gelling time slightly longer. |
| | | 0.8 | 50 | |
| C | 20% P188 | 0.05 | 10 | The most viscous of all |
| (37.53 ± 0.46 | 16% P407 | 0.1 | 20 | compositions tested. |
| | | 0.2 | 30 | T_{sol-gel} measured at 39° C. |
| | | 0.4 | 40 | (did not form gel at 37° C.). |
| | | 0.8 | 50 | |
| D | 5% P188 | 0.05 | 10 | The least viscous of all |
| (32.70 ± 0.26 | 18% P407 | 0.1 | 10 | compositions tested. |
| | | 0.2 | 20 | No remarkable difference |
| | | 0.4 | 20 | compared to Hydrogel |
| | | 0.8 | 20 | composition A on gelling time. |

*Tsol-gel values shown were determined as in Zhang K., Shi X. et al. 2014. Information is shown in Table 1.

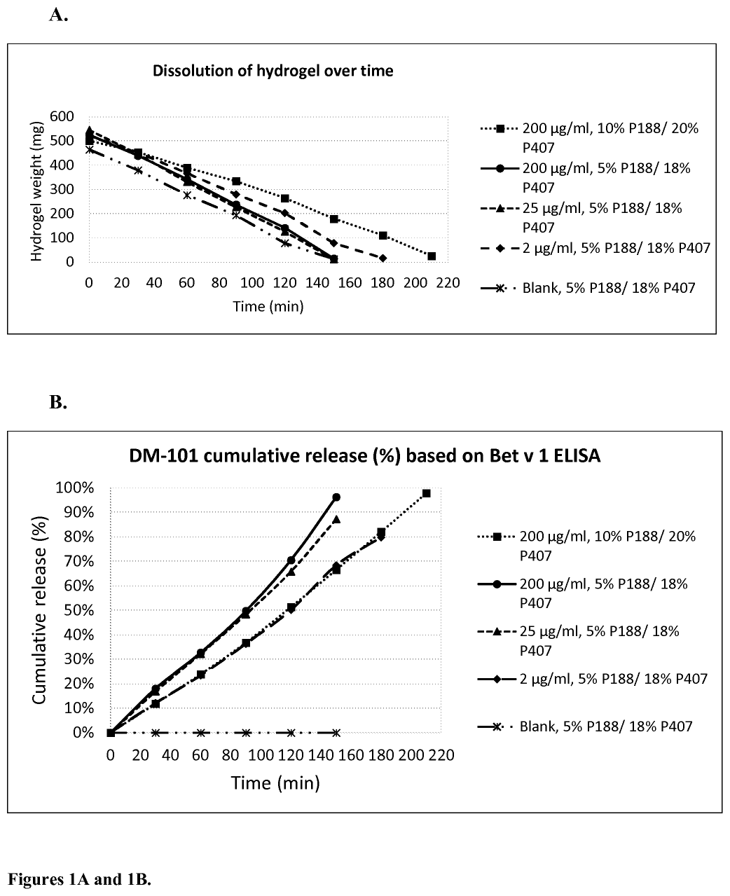

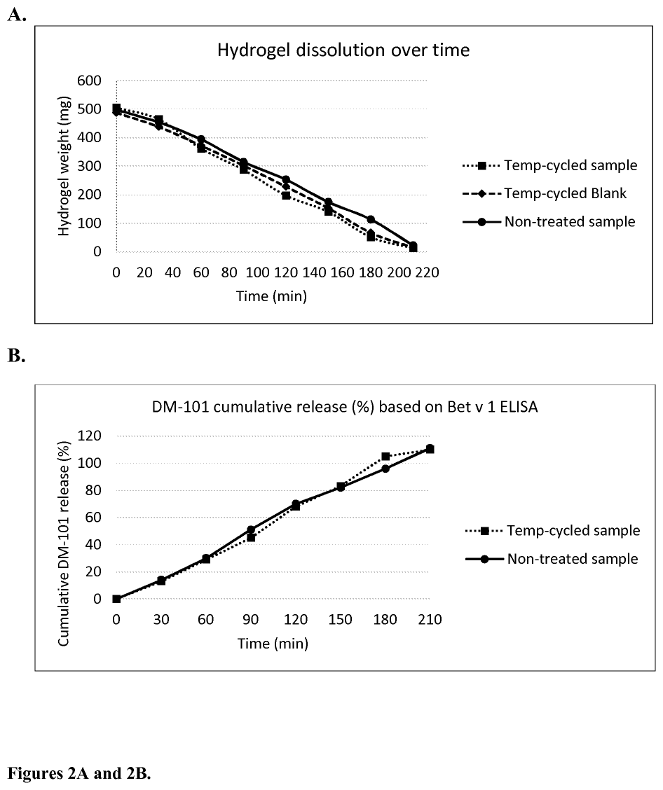

Protein release from 5% P188/18% P407 hydrogel. Results are shown in FIG. 1B and Table 2.

TABLE 2
10/20 hydrogel refers to 20% of P188 and 20% of P407.
5/18 hydrogel refers to 5% P188 and 18% P407.

| | | Released | DM-101 cumulative |
|---|---|---|---|
| DM-101 | Time | DM-101 per | release (%) based on |
| concentration | (min) | time point (%) | bet v 1 elisa |
| 200 μg/ml | 0 | | 0% |
| (in 10/20 | 30 | 12% | 12% |
| hydrogel) | 60 | 12% | 24% |
| | 90 | 13% | 37% |
| | 120 | 15% | 51% |
| | 150 | 15% | 66% |
| | 180 | 16% | 82% |
| | 210 | 16% | 98% |
| 200 μg/ml | 0 | | 0% |
| (in 5/18 | 30 | 18% | 18% |
| hydrogel) | 60 | 15% | 33% |
| | 90 | 17% | 50% |
| | 120 | 21% | 71% |
| | 150 | 26% | 96% |
| 25 μg/ml | 0 | | 0% |
| (in 5/18 | 30 | 17% | 17% |
| hydrogel) | 60 | 15% | 32% |
| | 90 | 16% | 48% |
| | 120 | 18% | 66% |
| | 150 | 21% | 87% |
| 2 μg/ml | 0 | | 0% |
| (in 5/18 | 30 | 12% | 12% |
| hydrogel) | 60 | 11% | 23% |
| | 90 | 13% | 36% |
| | 120 | 14% | 50% |
| | 150 | 18% | 68% |
| | 180 | 12% | 80% |

Viscosity and gelation of hydrogel. Testing the gel forming capability at 37° C. water bath. See Table 3.

TABLE 3

| | | | | Viscosity | Gelling | Gelation |
|---|---|---|---|---|---|---|
| | Sample | P188 | P407 | at 5/s | time (s) | temperature |
| # | name | (%) | (%) | (mPas) | at +37° C. | (′C.)* |
| 1 | 1/16 | 1 | 16 | 70 | 20 | |
| 2 | 2/16 | 2 | 16 | 73 | 35 | |
| 3 | 3/16 | 3 | 16 | 69 | 75 | 39 ± 0.50 |
| 4 | 3/17 | 3 | 17 | 103 | 25 | |
| 5 | 4/17 | 4 | 17 | 108 | 25 | |
| 6 | 5/17 | 5 | 17 | 104 | 40 | |
| 7 | 6/17 | 6 | 17 | 106 | 60 | |
| 8 | 7/17 | 7 | 17 | 106 | 90 | |
| 9 | 3/18 | 3 | 18 | 159 | 10 | 31.5 ± 1.00 |
| 10 | 4/18 | 4 | 18 | 143 | 15 | |
| 11 | 5/18 | 5 | 18 | 144 | 20 | 35.00 ± 0.50 |
| 12 | 6/18 | 6 | 18 | 146 | 20 | |
| 13 | 7/18 | 7 | 18 | 134 | 30 | |

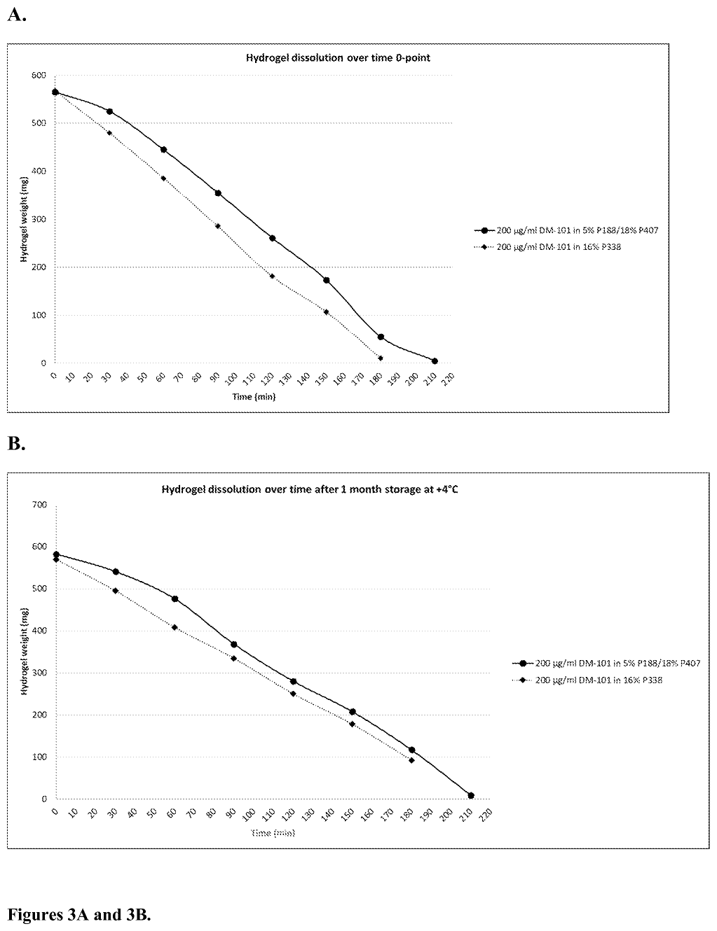

Reversibility capability of hydrogel. The reversibility capability of hydrogel was tested. The samples went through three gel-sol cycles prior analyzing the release of DM-101 by ELISA. See FIG. 2 for results.

Gel forming capability of P188 and P338 hydrogels. Gelation times at 37° C. and 33° C. are shown in Table 4.

TABLE 4

| | P188 | P338 | P407 | Gelation | Gelation |
|---|---|---|---|---|---|
| | % | % | % | time(s) | time (s) |
| Name | (w/w) | (w/w) | (w/w) | at +37° C. | at +33° C. |
| 0/16 | 0 | 16 | | 20 | 40 |
| 5/16 | 5 | 16 | | no gelation | no gelation |
| 0/17 | 0 | 17 | | 10 | 20 |
| 5/17 | 5 | 17 | | 45 | no gelation |
| 0/18 | 0 | 18 | | 10 | 15 |
| 1/18 | 1 | 18 | | 10 | 20 |
| 2/18 | 2 | 18 | | 15 | 30 |
| 3/18 | 3 | 18 | | 20 | 45 |
| 5/18 | 5 | 18 | | 20 | no gelation |
| 5/18 | 5 | | 18 | 15 | 40 |

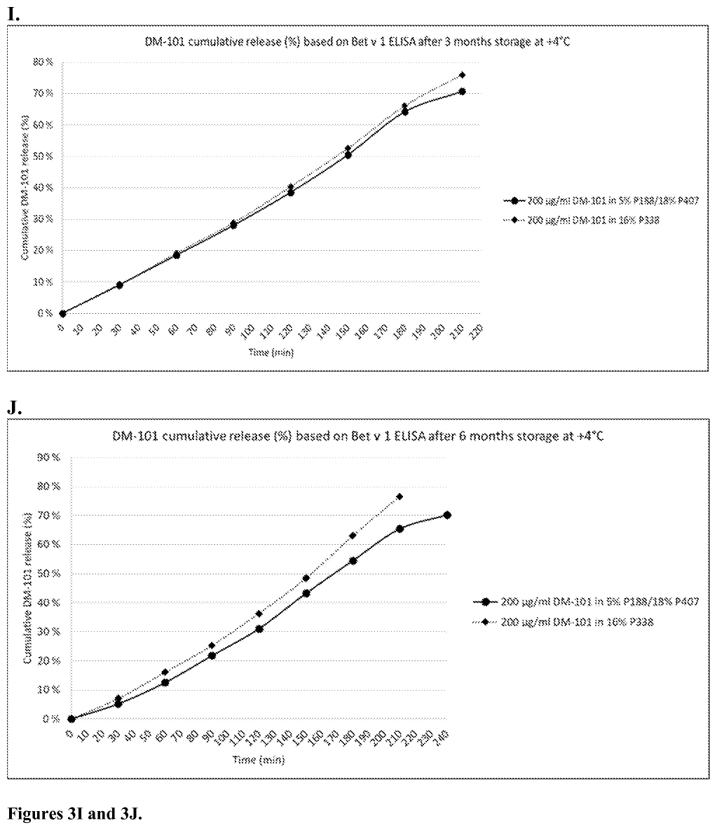

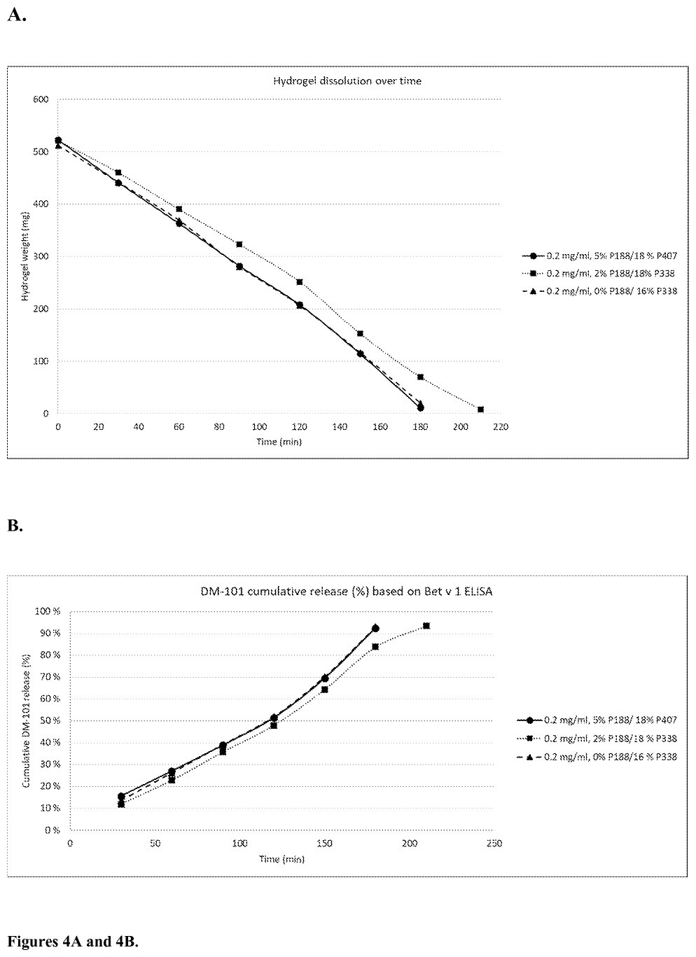

DM-101 release from P188/P407, P188/P338 and P338 hydrogels is shown in FIG. 4 and Table 5.

TABLE 5

| | Time | Released | DM-101 cumulative release (%) |
|---|---|---|---|
| Sample | (min) | DM-101 (%) | based on Bet v 1 ELISA |
| DM-101 | 0 | | 0% |
| 0.2 mg/ml, | 30 | 16% | 16% |
| 5% P188/ | 60 | 11% | 27% |
| 18% P407 | 90 | 12% | 39% |
| | 120 | 13% | 51% |
| | 150 | 18% | 70% |
| | 180 | 23% | 93% |
| DM-101 | 0 | | 0% |
| 0.2 mg/ml, | 30 | 12% | 12% |
| 2% P188/ | 60 | 11% | 23% |
| 18% P338 | 90 | 13% | 36% |
| | 120 | 12% | 48% |
| | 150 | 16% | 64% |
| | 180 | 19% | 84% |
| | 210 | 10% | 93% |
| DM-101 | 0 | | 0% |
| 0.2 mg/ml, | 30 | 13% | 13% |
| 0% P188/ | 60 | 13% | 26% |
| 16% P338 | 90 | 13% | 39% |
| | 120 | 13% | 52% |
| | 150 | 18% | 70% |
| | 180 | 23% | 93% |

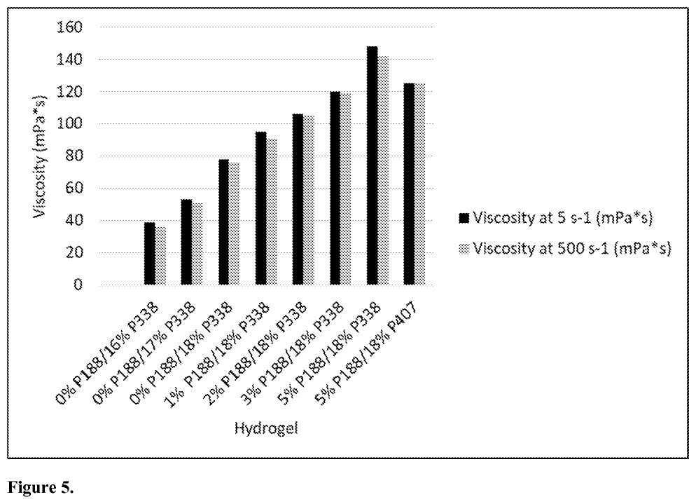

Viscosity of P188/P407, P188/P338 and P338 hydrogels is shown in FIG. 5.

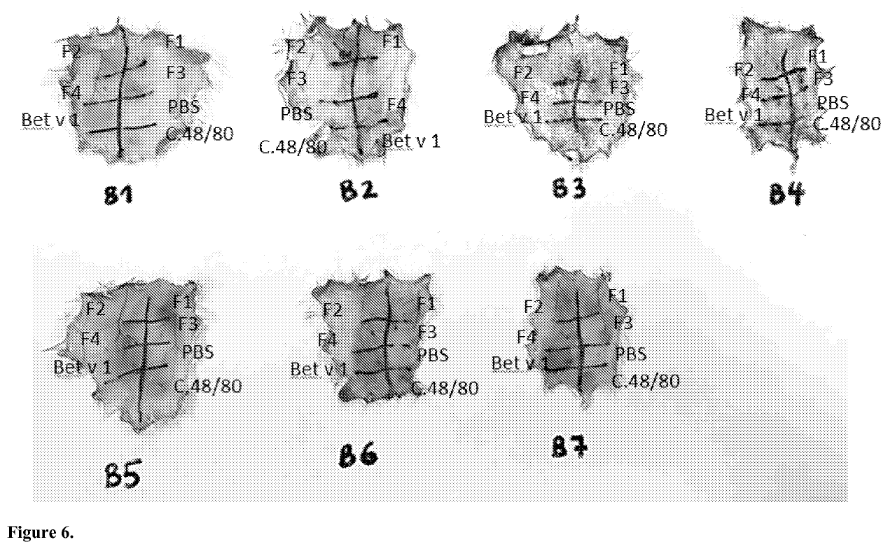

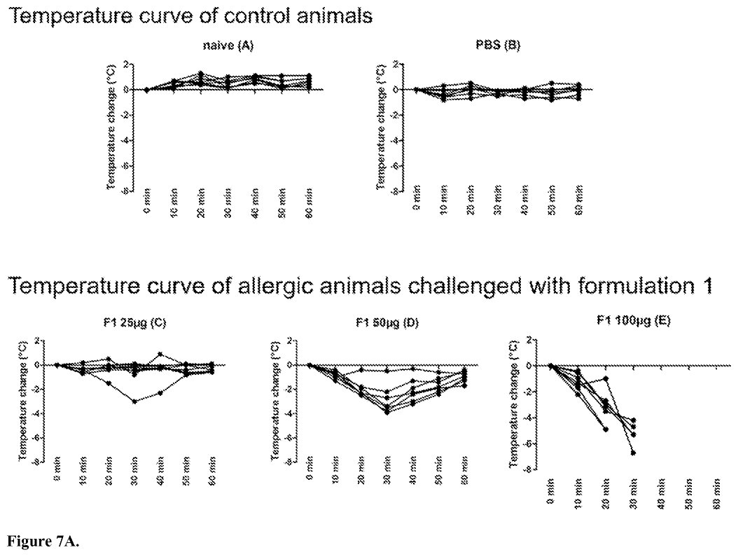

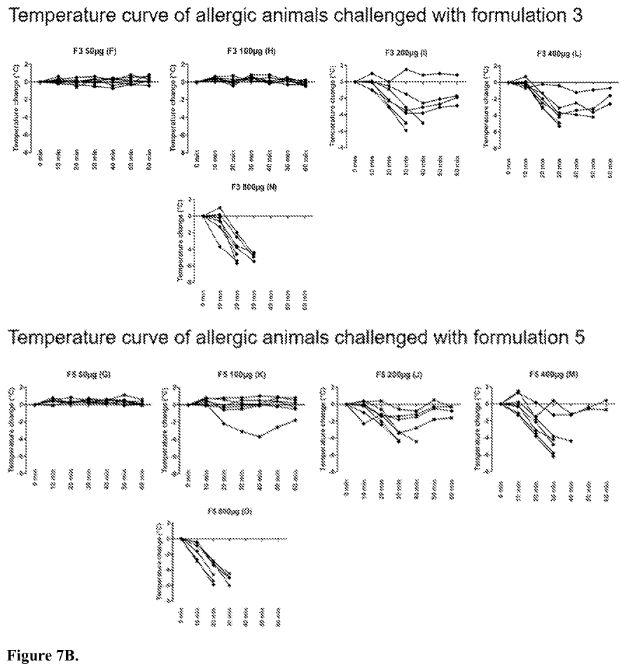

Allergenicity of formulations in mice. FIG. 6, skin prick test. FIG. 7, body temperature measurement after sc. challenge. The results are excellent and show that both poloxamer formulations 3 and 5 allow at least a fourfold higher DM-101 dose rate (i.e. at least 200-400 μg) compared to the buffer formulation 1 (25-100 μg).

### CONCLUSIONS

The aim of the present disclosure was to develop a novel formulation for use in allergen-tolerance promoting immunotherapy. The specific aims were 1) to protect the allergen from adsorption to surfaces (vials, syringes etc.) when delivered in low concentrations; 2) to protect the natural fold of the allergen from partial or total denaturation; 3) to obtain a depot effect in allergen exposure to the immune system over a few hours; and 5) to have the depot forming agent dissolving within a few hours. This has been achieved using poloxamers as stabilizing compound and their ability to form thermosensitive hydrogels as a depot agent.

In the experiments of the present disclosure, various compositions of poloxamers were tested for gelling temperature and gelling time to determine optimal concentrations for the desired use. The results are shown in Table 1 and FIG. 5. The viscosity determination is important as too viscous solutions at room temperature are difficult to deliver by hypodermic needles. Viscosity below 150 can easily be handled by 25 G needles which also was verified in practice in the experiments with mice. The gelling and viscosity of the present compositions were not affected by consecutive cycles of gelling and dissolution (FIG. 2).

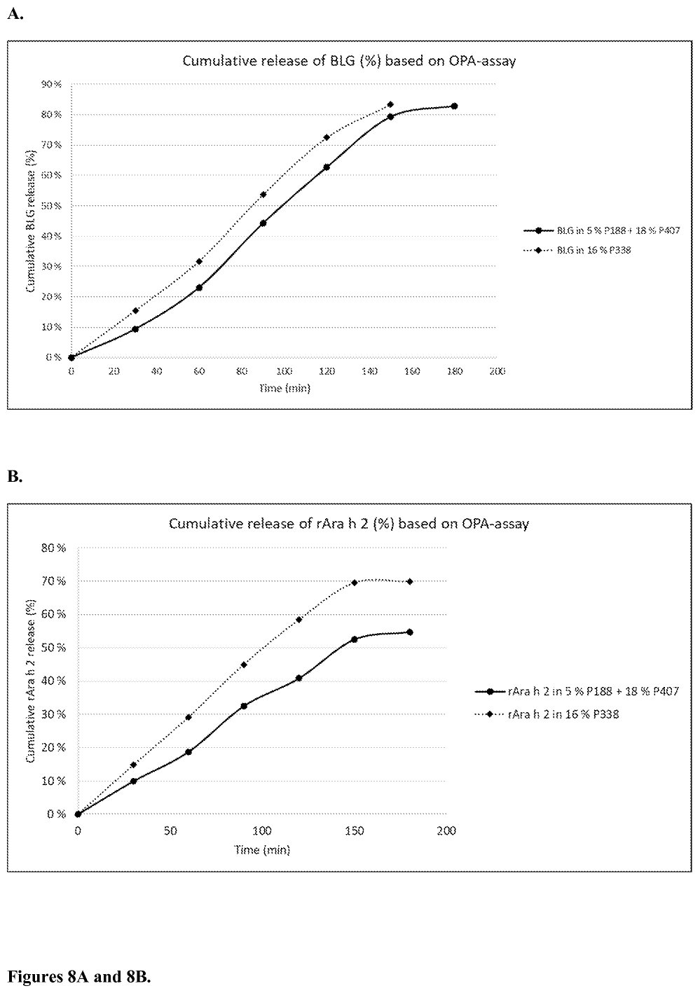

The release time for the allergens from the gel was measured in vitro as described in FIGS. 1B and 8B. Optimal release times (more than 60%-80% in 4 hours) were obtained with selected poloxamer mixtures. The release time was determined not only with the model hypoallergen DM-101 but also with the milk allergen BLG and with the peanut allergen Ara h 2 to show universality (FIG. 8B).

The time for the poloxamer compositions to dissolve was shown to be a few hours in vitro (FIGS. 1A and 8 A).

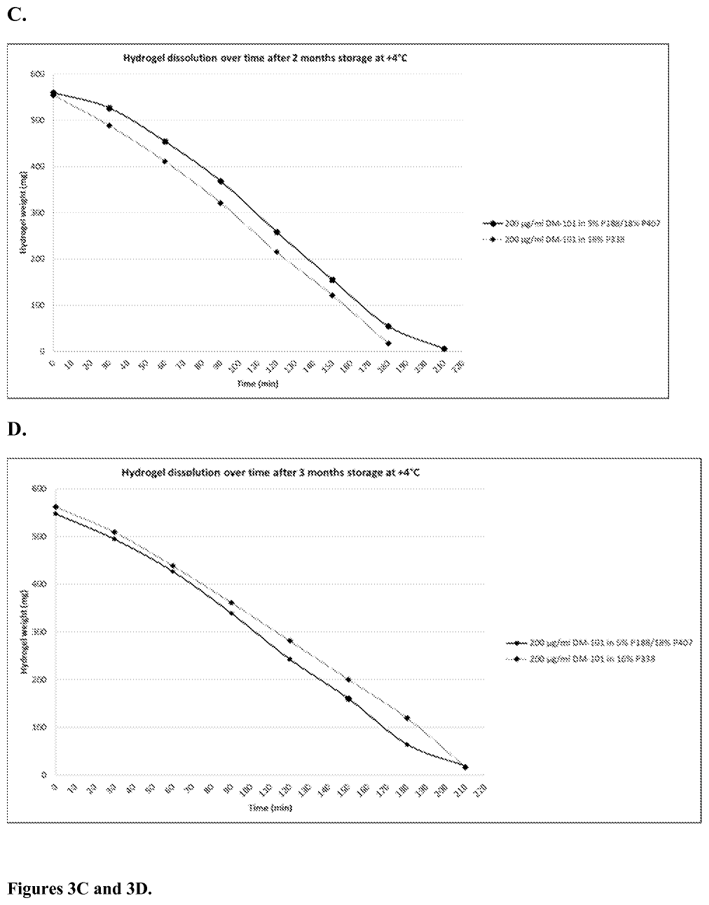

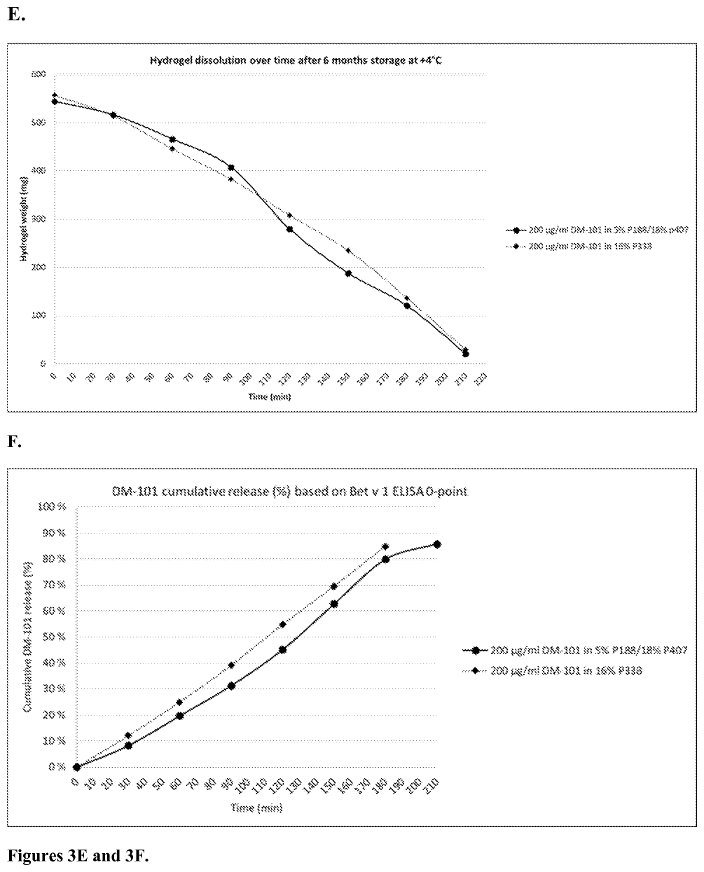

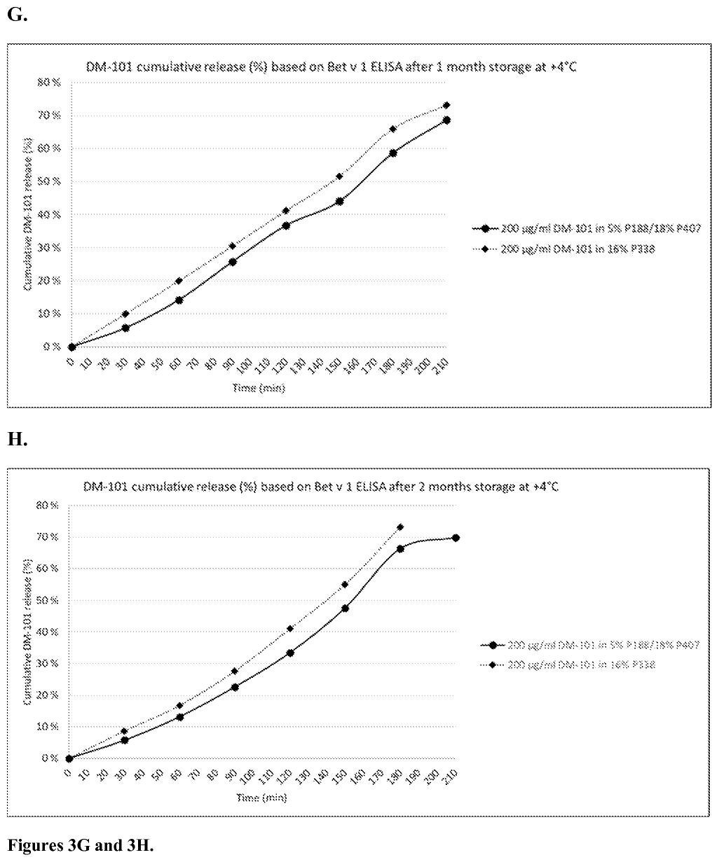

In FIG. 3, the results for measurements of storage stability of correctly folded DM-101 in different mixtures of poloxamers are shown. The amount of DM-101 was measured from the solution after storage at +4 for the indicated times (0-6 months), and the measurement was performed with an immunoassay which is fold sensitive, i.e. measures only correctly folded DM-101. The dissolution times (3A-3E) of the stored gels and the release of correctly folded DM-101 (3F-3J) were shown to be highly stable.

The controlled release of the allergen from the present poloxamer compositions decreases the adverse effects in sensitized mice, and hence allows for higher concentrations of hypoallergen or allergen in the immunotherapy. This is shown by the skin prick test results of FIG. 6. The results show that the DM-101 embedded in the poloxamer formulation of the present invention (sample F3) present less signs of inflammation compared to the DM-101 in plain buffer (sample F1).

In another test (see FIG. 7) for adverse allergic response sensitized mice were injected with the DM-101 agent in plain buffer (Formulation 1) and in poloxamer formulations (Formulations 3 and 5). The anaphylactic response is measured as the drop in body temperature of the mice. It can be seen that a significant drop in body temperature is seen when 50 μg DM-101 (Formulation 1) is injected in plain buffer while more than 200 μg are required to give a similar response with the poloxamer formulations (Formulations 3 ja 5).

Toxicity and safety. The biocompatible thermosensitive hydrogel compositions described herein are intended for subcutaneous or intramuscular injection of an active pharmaceutical ingredient (such as a protein for allergen immunotherapy). Hence the formulation must be safe and non-toxic in clinical use. Safety and toxicity were assessed in a 12-week repeated dose toxicity study in New Zealand White rabbits according to applicable international and European guidelines. This nonclinical toxicity study was conducted by a GLP compliant test facility for the 16.5% poloxamer 338 formulation with the DM-101 as active ingredient. The conclusion was that the administration of the poloxamer 338 formulation with DM-101 via subcutaneous route once a week for 12 weeks was well tolerated in New Zealand White rabbits at the studied DM-101 dose level of 0.1 mg/week. The dose volume administered to the rabbits (0.5 mL) was the same as intended to be used in the clinical trial. Thus, this toxicity study also demonstrated that the subcutaneous dosing of the excipient poloxamer 338 at the clinical dose level in rabbits was safe.

Thus this set of examples show that independent criteria for the aim of the formulation have been met.

### CITATION LIST

### Patent Literature

WO2009153414
WO2012143374
WO2019135027

### Non Patent Literature

Zhang K., Shi X. et al. 2015. Poloxamer-based in situ hydrogels for controlled delivery of hydrophilic macromolecules after intramuscular injection in rats. Drug Delivery; 22(3): 375-382.
Schöll I., Weissenböck A., Förster-Waldl E., Untersmayr E., Walter F., Willheim M., Boltz-Nitulescu G., Scheiner O., Gabor F. and Jensen-Jarolim E. 2004. Allergen-loaded biodegradable poly(D,L-lactic-co-glycolic) acid nanoparticles down-regulate an ongoing Th2 response in the BALB/c mouse model. Clin Exp Allergy 2004; 34:315-321.
Storni et al. Vaccine against peanut allergy based on engineered virus-like particles displaying single major peanut allergens. (2020) J Allergy Clin Immunol; 145:1240-53.

1. A biocompatible thermosensitive hydrogel composition for subcutaneous or intramuscular injection comprising a poloxamer, wherein said poloxamer is Poloxamer 338 or a mix thereof with Poloxamer 188, or said poloxamer is a mix of Poloxamer 407 and Poloxamer 188, said composition containing up to 25% (w/w) of said poloxamer so that said composition comprises 15-20% (w/w) of Poloxamer 338 or Poloxamer 407; and wherein said composition comprises a therapeutic protein or antigen embedded in said composition.

2. The biocompatible hydrogel composition according to claim 1, wherein said composition is in an injectable liquid state at 4-25° C. and forms a gel at 30-37° C. in 60 seconds, preferably in 30 seconds.

3. The biocompatible hydrogel composition according to claim 2, wherein up to 20% of said therapeutic protein or antigen is released from said composition in 30 minutes at 37° C.

4. The biocompatible hydrogel composition according to claim 3, wherein at least 60% of said therapeutic protein or antigen is released from said composition in 180-300 minutes at 37° C.

5. The biocompatible hydrogel composition according to claim 4, wherein said therapeutic protein or antigen is released from the composition in 240 minutes, preferably in 180 minutes.

6. The biocompatible hydrogel composition according to any of claims 1-5, wherein said therapeutic protein is an allergen, preferably a hypoallergen, more preferably a genetically engineered hypoallergen.

7. The biocompatible hydrogel composition according to claim 6, wherein said allergen is plant pollen, such as pollen from birch or timothy-grass, or a genetically engineered hypoallergen thereof, or wherein said allergen is an animal protein or a genetically engineered hypoallergen thereof.

8. The biocompatible hydrogel composition according to claim 7, wherein said allergen is birch allergen Bet v 1 or a genetically engineered hypoallergen thereof.

9. The biocompatible hydrogel composition according to claim 7, wherein said allergen is horse allergen Equ c 1, peanut allergen Ara h 2, or a genetically engineered hypoallergen thereof.

10. The biocompatible hydrogel composition according to any of claims 1-5, wherein said antigen comprises live virus, live bacteria, killed virus, killed bacteria, nucleic acids, protein subunits of infectious agents, or mixtures thereof.

11. The biocompatible hydrogel composition according to claim 10, wherein said composition is a vaccine.

12. The biocompatible hydrogel composition according to any of claims 1-11, wherein said composition comprises 4-6% (w/w) of Poloxamer 188 and 17-19% (w/w) of Poloxamer 407, preferably 5% (w/w) of Poloxamer 188 and 18% (w/w) of Poloxamer 407.

13. The biocompatible hydrogel composition according to any of claims 1-11, wherein said composition comprises 15-19% (w/w) of Poloxamer 338, preferably 15-17% (w/w) of Poloxamer 338, more preferably 16% of Poloxamer 338.

14. The biocompatible hydrogel composition according to any of claims 1-11, wherein said composition comprises 1-3% (w/w) of Poloxamer 188 and 15-19% (w/w) of Poloxamer 338, preferably 2% (w/w) of Poloxamer 188 and 18% (w/w) of Poloxamer 338.

15. The biocompatible hydrogel composition according to any of claims 1-14, wherein said composition comprises a sterile diluent such as water for injection, saline solution, fixed oils, polyethylene glycols, glycerine, propylene glycol or other synthetic solvents, antioxidants such as ascorbic acid or sodium bisulfite, chelating agents such as ethylenediaminetetraacetic acid (EDTA), preservatives, buffers such as acetates, citrates or phosphates.

16. The biocompatible hydrogel composition according to any of claims 1-15 further comprising an anti-IgE antibody and/or antihistamin.

17. The biocompatible hydrogel composition according to any of the claims 1-16 for use in allergen-tolerance promoting immunotherapy or the treatment of an autoimmune disease.

18. Method of treating an allergy or an autoimmune disease comprising administering an effective amount of the biocompatible thermosensitive hydrogel composition according to any of claims 1-16 to a patient in need thereof, wherein the administration is performed by a subcutaneous or intramuscular injection.

19. Use of the biocompatible thermosensitive hydrogel composition according to any of claims 1-16 for the manufacture of a medicament for the treatment of an allergy or an autoimmune disease.
